(19) United States
(12) Patent Application Publication
(10) Pub. No.: US 2010/0234841 A1
(43) Pub. Date: Sep. 16, 2010

(54) System and Method for Magnetic Tissue Ablation

(76) Inventors: Chris Butts, Evanston, IL (US); Forrest James Mertens, Denver, CO (US); Alex Cohen Herzick, San Francisco, CA (US); Krishanu Nandy, Evanston, IL (US); Mayte Zavaleta, Chicago, IL (US); Catherine Chen, St. Louis, MO (US); Joseph Thomas Breen, Rochester, MN (US); Colleen Fryer, Towson, MA (US)

Correspondence Address: Christopher Butts;#3D, 910 Washington, Evanston, IL, 60202, US

(21) Appl. No.: 12/721,137

(22) Filed: Mar. 10, 2010

Related U.S. Application Data
(60) Provisional application No. 61/158,772, filed on Mar. 10, 2009.

(51) Int. Cl. A61B 18/14

(52) U.S. Cl. 606/41

(57) ABSTRACT

Herein it is described a magnetic medical device, and a system and method in which the magnetic medical device is implemented, that enables bipolar, minimally invasive, tissue ablation. The magnetic medical device may include magnetic means for magnetically coupling the medical device to a second medical device, wherein the magnetic medical devices may be separated by the tissue to be ablated. The magnetic medical device may also include electrical means for transferring electrical energy between the magnetic medical device and the second medical device. The magnetic medical device may further include control means whereby the physical location of the magnetic medical device may be controlled and thereby, as a result of the magnetic coupling, the physical location of the second magnetic medical device may also be controlled.

### RELATED APPLICATIONS

This application claims priority from U.S. Provisional Patent Application No. 61/158,772, filed Mar. 9, 2009, incorporated herein by reference in its entirety.

### BACKGROUND

Atrial fibrillation (AF) is the most common persistent cardiac arrhythmia, affecting an estimated 2.2 million people in the United States and 4.5 million people in Europe. The prevalence of atrial fibrillation increases with increasing age—it occurs in 3.8 percent of people 60 years and older, and in 9.0 percent of people 80 years and older. It is estimated that 5.6 million people in the United States will be affected by 2050, with approximately 160,000 new cases diagnosed annually. Additionally, as atrial fibrillation is frequently asymptomatic, it is anticipated that the true incidence of atrial fibrillation may be double that of previously reported rates.

AF is characterized by rapid, disorganized atrial electrical activation, leading to uncoordinated atrial contraction and blood stasis within the atria. This stasis can result in clot formation in the atria and secondary emboli that can travel throughout the arterial vasculature, including the cerebral circulation, where their blockage of blood flow can result in ischemic stroke. The most common origin of the abnormal electrical activation is in the pulmonary veins, where over 90% of ectopic foci are located. AF is a significant risk factor for ischemic stroke, with an annual risk of 1.3%. It is associated with a five-fold increased risk of stroke or embolism compared to patients without atrial fibrillation. At least 15% of strokes in the United States are attributed to atrial fibrillation.

Given the poor efficacy rates of pharmacologic AF treatments, AF surgery has emerged as a viable treatment alternative. The purpose of AF surgery is to create reliable conduction blockage to eliminate macro-reentry electrical circuits in the atria. Catheter and surgical ablation target pulmonary vein isolation to achieve this goal and promote atrioventricular synchrony and hemodynamic improvement, including quality of life improvement. A number of different lesion patterns have been practiced, including bilateral atrial lesions, isolated atrial lesions, septal lesions, left atrial appendage lesions, and mitral annulus lesions. While only some of the lesions sets have proven effective in blocking circuitry, the left atrial appendage and mitral annulus with the pulmonary vein lesion set continues to be the most commonly performed lesions. Research has established that a curative approach for persistent and permanent AF should target structures and substrates outside the pulmonary vein region.

Until the last decade, the Cox-Maze III surgical procedure (“Maze”) was the only surgical alternative to pharmacologic treatment. While this surgery is the “gold standard” in AF treatment, with reported success rates of 93% at 8.5-year follow-up points and associated long term stroke rates of less than 1%, it requires cardiopulmonary bypass, is technically complex, very invasive (open heart, median sternotomy), and often requires permanent pacing due to the loss of atrial transport function. As a result, the procedure is not widely used outside of concomitant open heart surgeries, such as coronary artery bypass grafts (CABG) or mitral valve replacements.

Given the complex and invasive nature of the Maze, minimally invasive surgery and percutaneous catheter-based treatments have emerged as alternatives. Though current treatment guidelines still recommend surgery as a second line alternative to AADs, primarily due to the lack of quality clinical data available to assess long term efficacy of these newer procedures, shorter term efficacy rates of minimally invasive surgery and percutaneous catheter treatments are much improved over pharmacologic treatments, and invasiveness and complexity is reduced as compared to the Maze. Various research reports and trials have yielded efficacy rates for surgical and catheter treatments from 40%-90%; however, adjusted efficacy rates (controlling for repeat procedures, continued AAD therapy, and post-operative cardioversion) range from 40%-60%. These less invasive procedures struggle to consistently deliver transmural lesions, resulting in unblocked reentry circuits in the heart.

These procedures also need real time monitoring to guide physician action with regard to lesion creation. Technically, a transmural lesion is one that extends from the epicardial surface of the heart fully through the myocardium to the endocardial surface of the heart. While real time observation is difficult, technologies that monitor heat, conductance, impedance, and time help direct physician action.

Currently, catheter techniques are primarily unipolar solutions. These solutions are unable to consistently produce transmural lesions and have been reported to cause significant complications, including esophageal fistula, stroke, cardiac tamponade, and pulmonary vein stenosis. For example, in radiofrequency unipolar catheters, electrical current flows from the distal tip through the heart tissue and then through the body to a grounding pad. As a result, patients experience a rapid decline, due to dissipation, of energy delivered to the deeper heart tissues, often resulting in an inability to create transmural lesions.

The typical catheter ablation can take anywhere from 3-10 hours for an experienced electrophysiology cardiologist (EP). The procedure is technically difficult and labor intensive, and only high volume centers are reporting the best success rates. Repeat procedures are needed on 10-40% of patients and data are scarce on clinical outcomes greater than 2 years post-procedure.

To address low efficacy rates, newer ablation catheters have tested saline irrigated catheters. The saline cools the electrode tip to decrease the dissipation of heat and, as a result, these catheters create deeper and more uniform lesions. However, this solution is associated with a greater risk of tissue eruption and cardiac perforation. In addition, recent advancements in high quality mapping of ectopic foci allows for more targeted ablation sites, speeding the procedure and potentially improving efficacy.

Minimally invasive ablation surgery has emerged even more recently than catheter ablation as a treatment alternative. The main advantage of minimally invasive surgery is that physicians can apply bipolar energy, whereas catheters are currently constrained to unipolar energy. Bipolar energy is advantageous because the ablation line (energy current) is confined within the “jaws” (or points) of the ablation device. As a result, lesions lines are thin and discrete, and heat sink and collateral tissue damage are minimized. The bipolar devices can also better measure the characteristics of the tissue at the target area, and ablation points can be completed in less than 10 seconds; one study claimed to have achieved PV isolation in ten minutes. Though the clamp devices boast faster ablation times than unipolar catheters, an important shortcoming in the clamp technology is that the clamps allow the bipolar current to avoid ablation of high resistance tissue. The entire 5 cm clamp surface provides a large range of exit points for the current.

Other beneficial characteristics of this procedure include no fluoroscopy, shorter operating times, uncommon proarrythmias, reliable treatment of autonomic nerves, and fewer complications. While video probes replace fluoroscopy, the advanced mapping of ectopic foci that is used to guide catheter ablation is not yet available for these epicardial solutions. However, some physicians are mapping ganglionic plexi activity to confirm the isolation of the errant impulses. Minimally invasive surgery can also benefit those patients with a contraindication to anticoagulant drug therapy, a necessary therapy to prevent clotting in catheter procedures. Physicians can also remove the left atrial appendage using this minimally invasive approach. Finally, most of the procedures and products can also be conducted in an open heart concomitant setting.

### BRIEF SUMMARY

Herein it is described a magnetic medical device, and a system and method in which the magnetic medical device is implemented, that enables bipolar, minimally invasive, tissue ablation. The magnetic medical device may include magnetic means for magnetically coupling the medical device to a second medical device, wherein the magnetic medical devices may be separated by the tissue to be ablated. The magnetic medical device may also include electrical means for transferring electrical energy between the magnetic medical device and the second medical device. The magnetic medical device may further include control means whereby the physical location of the magnetic medical device may be controlled and thereby, as a result of the magnetic coupling, the physical location of the second magnetic medical device may also be controlled.

### BRIEF DESCRIPTION OF THE DRAWINGS

FIG. 1 depicts an exemplary ablation system.

FIG. 2 depicts an exemplary medical probe.

FIG. 3 depicts an exemplary distal end of a catheter.

FIG. 4 depicts exemplary magnetic and electrical means.

FIG. 5 depicts an exemplary medical probe.

FIG. 6 depicts an exemplary catheter.

### DETAILED DESCRIPTION

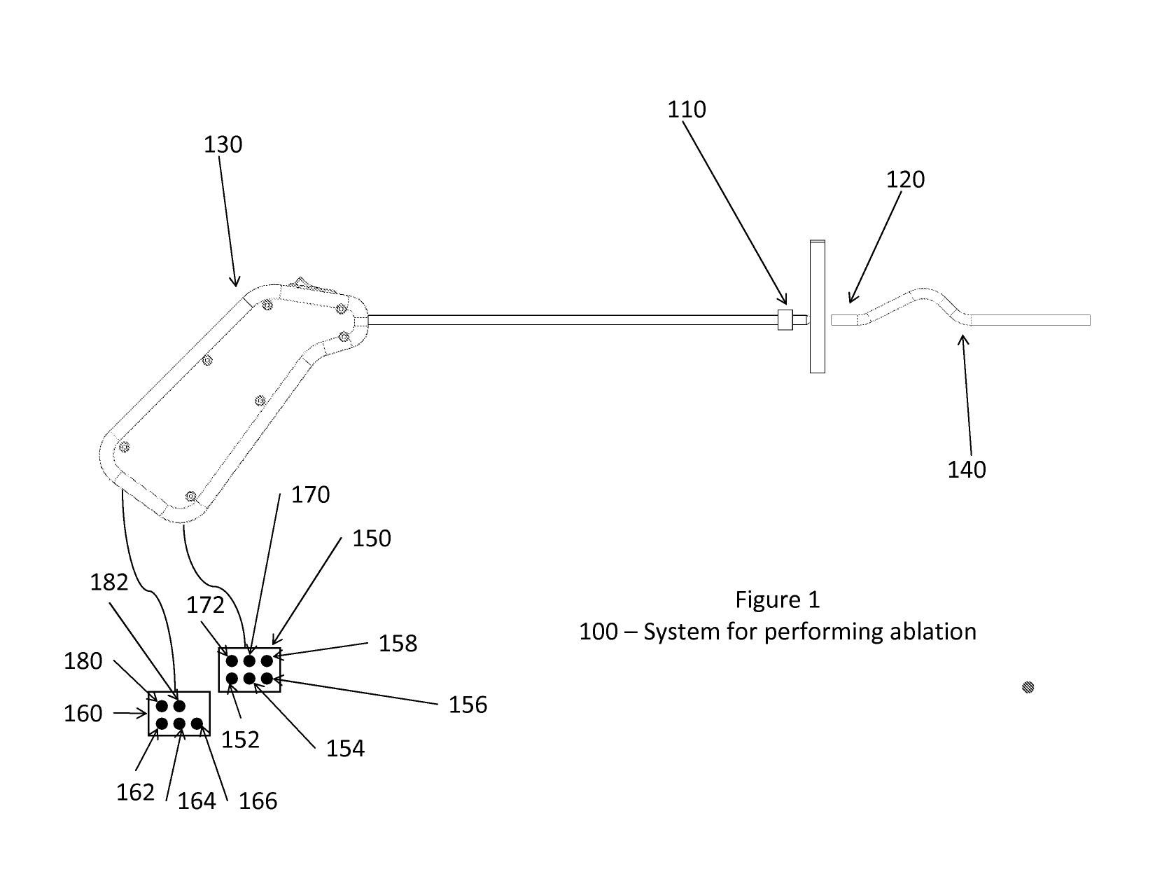

FIG. 1. is an exemplary ablation system. With reference to FIG. 1, in one embodiment the system for performing tissue ablation 100 comprises a first magnetic medical device 110 and a second magnetic medical device 120. The first magnetic medical device 110 and the second magnetic medical device 120 are configured to be magnetically and electrically coupled. In one embodiment the first magnetic medical device 110 may be attached to the distal end of a medical probe 130, and the second magnetic medical device 120 may be attached to the distal end of a catheter 140. The system for performing tissue ablation 100 may include an ablation power supply 150 configured to provide electrical energy to at least one of the first magnetic medical device 110 or the second magnetic medical device 120. Additionally, the system for performing tissue ablation 100 may include a magnetic power supply 160 configured to power magnetic means of the magnetic medical device 110, wherein the magnetic means comprise an electro-magnet.

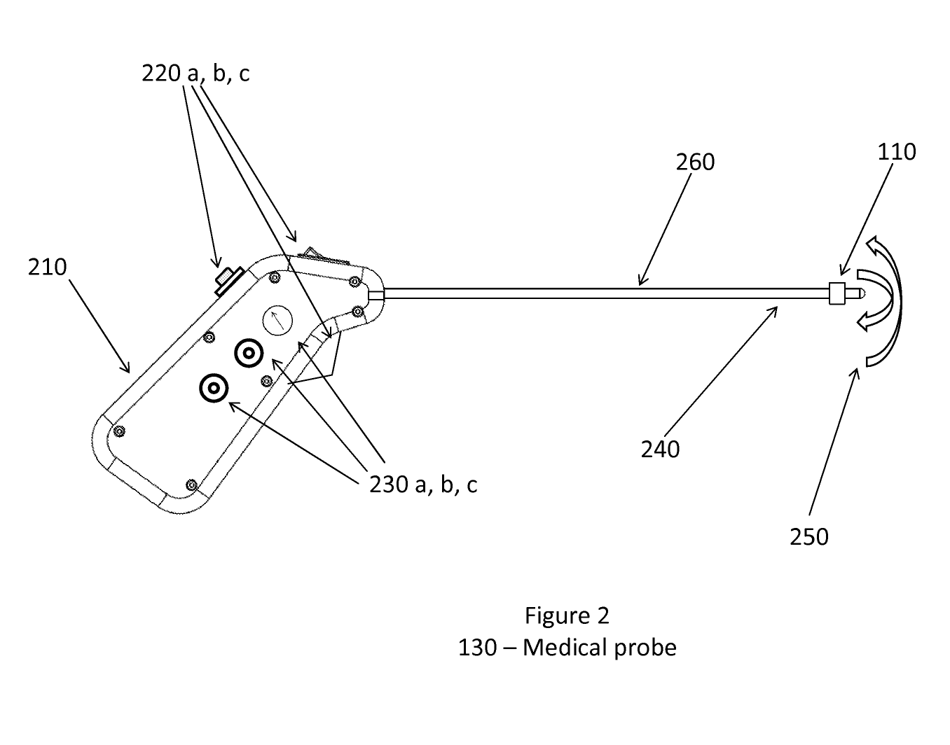

In one embodiment at least one of the first magnetic medical device 110 and the second magnetic medical device 120 may be configured to be connected to the distal end of a medical probe 130. With reference to FIG. 2, a magnetic medical device 110 may be connected to the distal end 240 of a medical probe 130 configured to be inserted into a thoracic cavity. Proximal end 210 of medical probe 130 may include an input device 220 for controlling modes of operation of the magnetic medical device. Further, proximal end 210 of medical probe 130 may include multiple input devices, for example 220a,b,c, as desired to control multiple modes of operation of the magnetic medical device. Proximal end 210 of medical probe 130 may also include an indication device 230 for indicating, for example, an operating characteristic of the magnetic device or a characteristic of the environment in which the magnetic medical device is being deployed. Further, proximal end 210 of medical probe 130 may include multiple indication devices, for example 230a,b,c, as desired for indicating multiple operating characteristics of the magnetic device in addition to multiple characteristics of the environment in which the magnetic medical device is deployed. Distal end 240 of the medical probe 130 may include a magnetic medical device 110 and control means 250 for controlling the physical location of the magnetic medical device 110. The medical probe also includes an extendable region 260 for deploying the distal end 240.

Proximal end 210 of the medical probe 130 may take the form of an ergonomic handle. Proximal end 210 may take other forms such as a general control platform. Input devices 220 may be placed on the proximal end 210 such that they are easily utilized. For example, the input devices may be placed such that they are easily accessed by a user's thumb. Alternatively, the input devices may be placed such that they are easily accessed by a user's forefinger. Indication devices 230 may be placed on the proximal end such that they are easily visible by a user. It should be noted that the specific placement of the input devices 220 and indication devices 230 depicted is for explanation only and therefore should not be taken to be limiting.

Input devices 220a,b,c may be implemented such that their utilization controls modes of operation of the magnetic medical device. For example, an input device may turn on magnetic coupling means of the magnetic medical device 110 when pressed, and turn off the magnetic coupling means when pressed a second time. Another input device may turn on electrical energy means of the magnetic medical device 110 when pressed, and turn off the electrical energy means if pressed a second time. The input devices may be able to variably control modes of operation of the magnetic medical device. For example, an input device may be a rotatable knob that varies the intensity of either the magnetic coupling means or the electrical energy means in response to the user rotating the knob. Other modes of operation may be controlled in response the utilization of input devices 220.

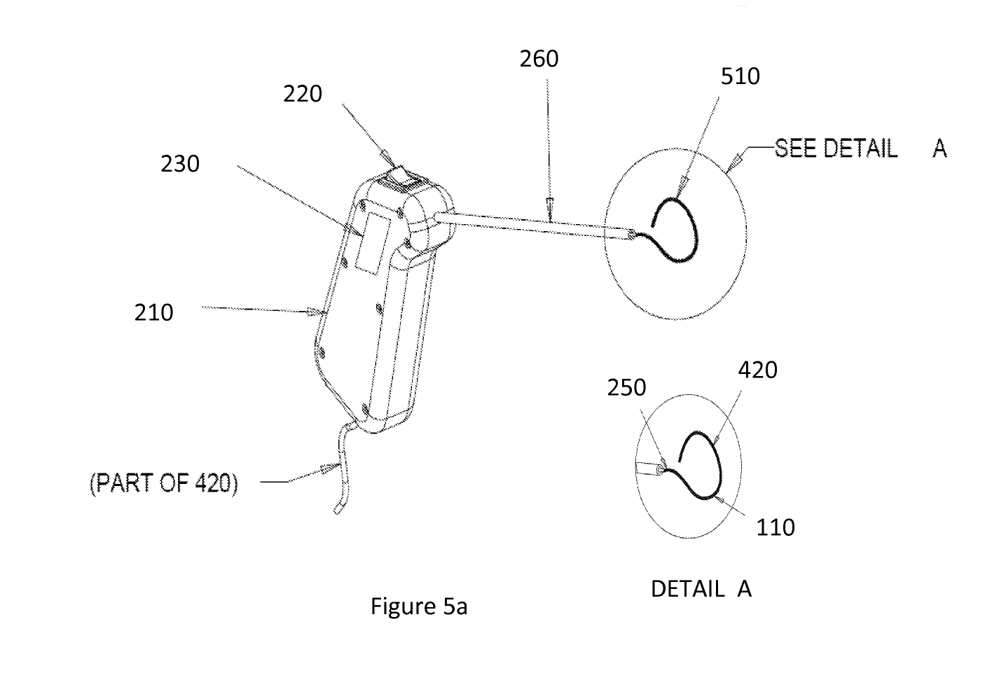

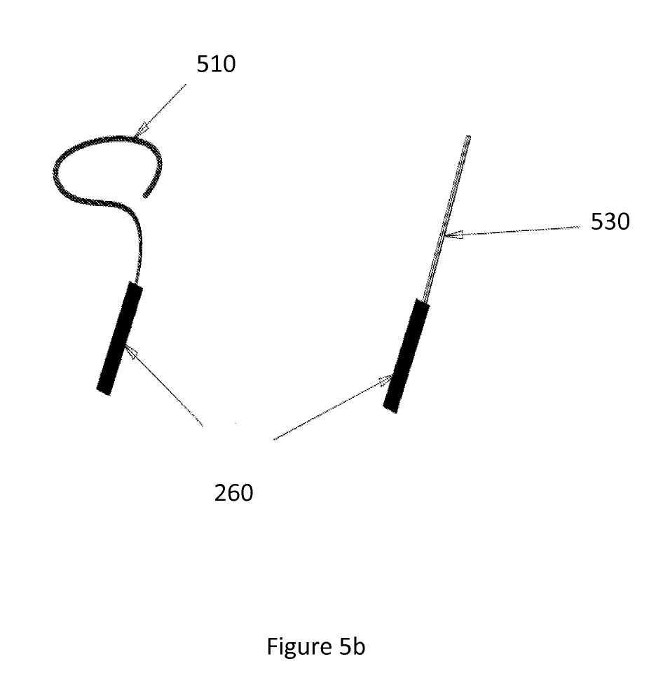

Distal end 240 of the medical probe 130 may take on any number of forms for the purpose of implementing control means 250 for controlling the physical location of the magnetic medical device 110. In one embodiment, control means 250 may include electro-mechanical means for actively guiding the physical location of the magnetic medical device. For example, electro-mechanical means may include receiving an electrical signal provided from an input device 220, and responsively physically moving the magnetic medical device. The physical movement of the magnetic medical device 110 may include any number of ranges of movement including moving linearly, vertically, circularly, or any combination thereof. The physical movement of the magnetic medical device may also include rotating the magnetic medical device 110 about the distal end 240 of the medical probe 130. In another embodiment, control means 250 may include a circular implement at the end of the extendable region 260, as depicted in FIG. 5.

With reference to FIG. 5a, the circular implement 510 may be an extension of the extendable region 260 of the medical probe. In one embodiment the circular implement may be hollow so that the magnetic medical device (not pictured) may freely move within and throughout the circular implement. In such an embodiment, the control means 250 may include a physical connection such as a pull wire (not pictured) that connects the magnetic medical device 110 at the distal end of the medical probe to an input device at the proximal end of the medical probe. A user may, by way of the pull wire, move the magnetic medical device 120 throughout the circular implement. With reference to FIG. 5b, the circular implement may be configured to be capable of being straightened 530. Straightening the circular implement may provide benefit in the deployment of the medical probe in certain situations. For example, in deploying the circular implement it may be beneficial to control the circumference or diameter of the circular implement 510 deployed. It may also be beneficial to control or manipulate the exact form that the circular implement 510 takes. Circular implement 510 may actually not take the form of a perfect circle, but may form any shape of a closed path necessary in particular applications.

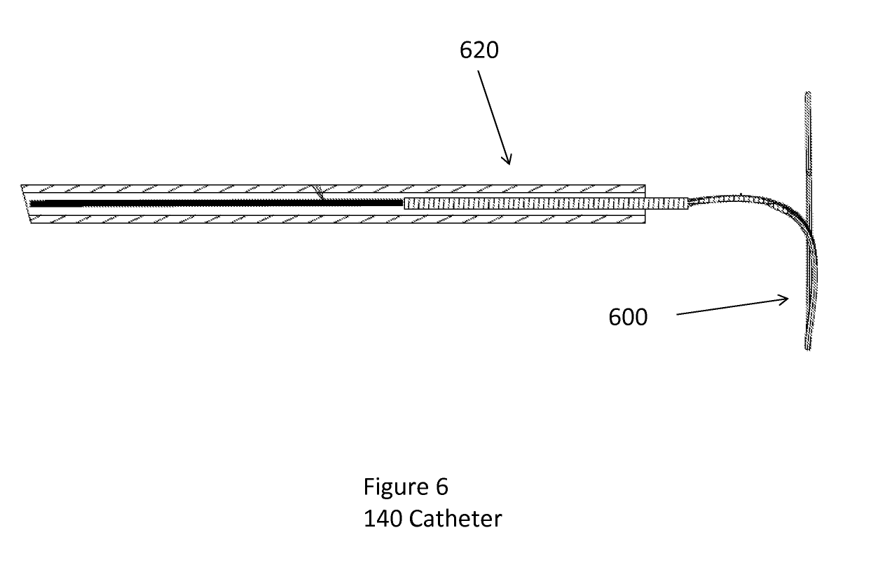

With reference to FIG. 6 a similar circular implement 600 may be deployed at the distal end 620 of a catheter 140. In one embodiment, the circular implement 600 would be deployed through the catheter in a straightened fashion similar to 530 described above and once the circular implement 600 leaves the distal end 620 of the catheter 140, it acquires its circular shape. Circular implement 600 may take any of the shapes discussed with reference to circular implement 510 above.

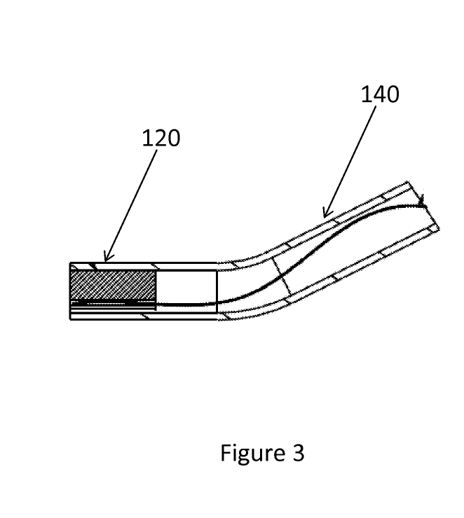

FIG. 3. shows magnetic medical device 120 deployed generally at the distal end of a catheter 140. Catheter 140 may have the magnetic medical device 120 rigidly fixed to its distal end, or the magnetic medical device 120 may be remotely deployed from within the catheter once the catheter has reached a desire position within a patient's body.

Ablation power supply 150 may be any of a variety of medical power supplies. For example, ablation power supply 150 may include means to vary the magnitude of the voltage applied 152, the magnitude of the current applied 154, the frequency of the power supplied 156, and the duty cycle of the power supplied 158. Power supply 150 may also include connections 170 and 172 for connecting the electrical means 420 of magnetic medical device 400 to the ablation power supply.

Magnetic power supply 160 may be any power supply capable of powering an electro-magnet. For example, magnetic power supply 160, may include means to vary the magnitude of the voltage applied 162, the magnitude of the current applied 164, and the frequency of the power supplied 166. Power supply 160 may also include connections 180 and 182 for connecting the magnetic means 410 of magnetic medical device 110 to the magnetic power supply.

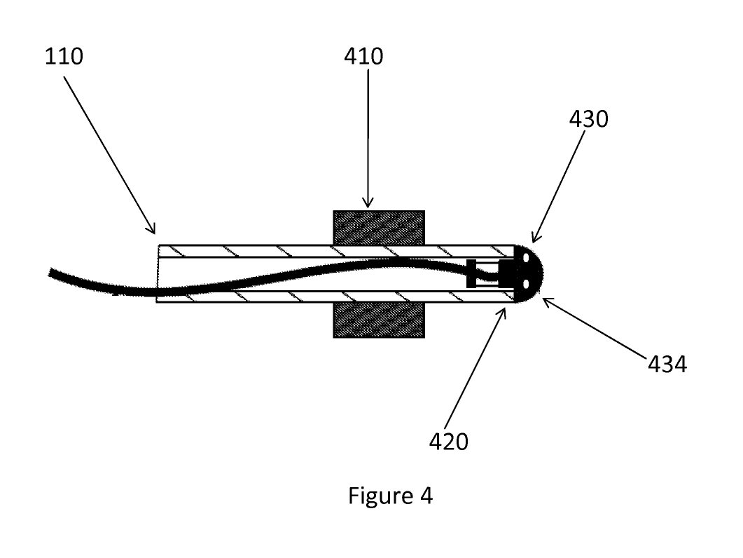

With reference to FIG. 4, magnetic medical device 110 includes magnetic means 410 for magnetic coupling and electrical means 420 for transferring electrical energy. The electrical means 420 of the magnetic medical device 110 may also include a plurality of sensing devices including a magnetic coupling detector 430 or an ablation detector 434.

Magnetic means 410 may be any magnetic material or electro-magnet capable of magnetically coupling the magnetic medical device 110 to a second magnetic medical device 120. In one embodiment magnetic means 410 may be a permanent magnet such as a rare earth magnet. In another embodiment magnetic means 410 may be an electro-magnet driven by magnetic power supply 160. Generally, magnetic means 410 may be configured such that the magnetic medical device may be coupled to a second magnetic medical device in such a way that electrical energy can be transferred between electrical means 420 of the first and second magnetic medical devices.

Electrical means 420 may be any electrically conductive material capable of transferring electrical energy from the magnetic medical device 110 to a second magnetic medical device. It should be noted that although the particular embodiment discussed refers specifically to electrical means for performing ablation, ablation may be performed in any number of additional ways known in the art. Such additional ways may be by way of electro-magnetic energy,

Magnetic coupling detector 430 may comprise an ohmmeter that measures resistance between the electrical means 420 of the magnetic medical device 110 and the electrical means of a second magnetic medical device. When a magnetic medical device 110 is magnetically coupled to a second magnetic medical device with tissue in between, a measured resistance, along with known electrical characteristics of the tissue can be used according to a logic set to determine that the magnetic medical devices are properly magnetically coupled. For example, it may be determined that magnetic medical devices are properly magnetically coupled when the measured resistance is at some minimum value. Alternatively, it may be determined that magnetic medical devices are properly magnetically coupled when the measured resistance is within some pre-defined threshold of the known resistance of the tissue being ablated.

Ablation detector 434 may comprise an impedance meter to measure the impedance across the tissue between a magnetic medical device 110 that is magnetically coupled to a second magnetic medical device. A measured impedance, along with known electrical characteristics of ablated tissue can be used according to a logic set to determine that the tissue has been ablated. For example, it may be determined that the tissue has been ablated if the measured impedance is greater than some threshold value. Alternatively, it may be determined that the tissue has been ablated when the measured impedance is within some pre-defined threshold of the impedance desired for the ablated tissue. Ablation detector 434 may also take the form of a tissue surface temperature measuring device, such as a thermal couple, that measures the surface temperature of the tissue.

To carry out the ablation procedure in treatment of atrial-fibrilation the distal end of catheter 140 may be floated to the right atrium of a patient by way of the femoral vein using known techniques. The distal end of medical probe 130 may be placed in the thoracic cavity by way of a trocar using known techniques. Additional ports providing access to the thoracic cavity to aid in visualization and or control of the distal end of medical probe 130 may also be utilized. One the catheter 140 and medical probe 130 are positioned, endocardially and epicardially respectfully, magnetic means 410 may be engaged so that the medical devices 110 and 120 become magnetically coupled. Once the medical devices 110 and 120 are magnetically coupled, they will be separated by the heart wall. Accordingly control means 250 may be initiated to control the physical location of both medical devices. At any point, electrical means 420 may be engaged to induce an electrical current across the heart wall and between magnetic medical devices 110 and 120, thus ablating the heart tissue between the medical devices. Various system parameters that may be employed in ablating tissue, such as electrical energy characteristics or optimal force to be exerted by the medical devices on the heart wall, are known in the art and may be utilized in the present system according to the desired effect.

We claim:

1. An ablation system comprising:
a first magnetic medical device;
a second magnetic medical device;
electrical means, attached to each of the first magnetic medical device and the second magnetic medical device, for transferring ablation energy between the first medical device and the second medical device;
control means for controlling the physical location of at least one of the first magnetic medical device and the second magnetic medical device; and
magnetic means attached to each of the first magnetic medical device and the second magnetic medical device for magnetically coupling the first magnetic medical device to the second magnetic medical device, such that the physical location of both the first magnetic medical device and the second magnetic medical device may be controlled by the control means.

2. The ablation system of claim 1, wherein the first magnetic medical device is connected to a distal end of a catheter.

3. The ablation system of claim 1, wherein the second magnetic medical device is connected to a distal end of a medical probe.

4. The ablation system of claim 1, wherein the first magnetic medical device is connected to a distal end of a catheter, and wherein the second magnetic medical device is connected to a distal end of a medical probe.

5. The ablation system of claim 4, wherein the second magnetic medical device connected to the distal end of the medical probe comprises the second magnetic medical device positioned within the distal end of the medical probe, and the control means is configured to control the physical location of the second magnetic medical device by moving the second magnetic medical device within the distal end of the medical probe.

6. The ablation system of claim 5, wherein the distal end of the medical probe is configured to form a closed path.

7. The ablation system of claim 6, wherein the control means comprises a pull wire connected to the second magnetic medical device and extending to a proximal end of the medical probe.

8. The ablation system of claim 4, wherein the first magnetic medical device connected to the distal end of the catheter comprises the first magnetic medical device positioned within the distal end of the catheter, and the control means is configured to control the physical location of the first magnetic medical device by moving the first magnetic medical device within the distal end of the catheter.

9. The ablation system of claim 8, wherein the distal end of the catheter is configured to form a closed path.

10. The ablation system of claim 9, wherein the control means comprises a pull wire connected to the second magnetic medical device and extending to a proximal end of the medical probe.
